(19) United States
(12) Patent Application Publication
(10) Pub. No.: US 2023/0135608 A1
(43) Pub. Date: May 4, 2023

(54) PHARMACEUTICAL COMPOSITION CONTAINING DIMETHYL FUMARATE AS AN ACTIVE INGREDIENT PROVIDES A SPECIFIC PHARMACOKINETIC PARAMETER

(71) Applicant: CURACLE CO., LTD., Seoul (KR)

(72) Inventors: Ji-Hye Kang, Seoul (KR); Myung-Hwa Kim, Seoul (KR); Heewon Seo, Seoul (KR); Jung-In Pyo, Seoul (KR)

(73) Assignee: CURACLE CO., LTD., Seoul (KR)

(21) Appl. No.: 17/885,388

(22) Filed: Aug. 10, 2022

Related U.S. Application Data
(63) Continuation of application No. PCT/KR2022/004172, filed on Mar. 24, 2022.

(30) Foreign Application Priority Data
Mar. 25, 2021 (KR) 10-2021-0038418

(51) Int. Cl. A61K 31/225; A61K 9/20; A61K 9/28; A61P 13/12; A61P 3/10

(52) U.S. Cl. CPC A61K 31/225; A61K 9/2018; A61K 9/2054; A61K 9/2027; A61K 9/2009; A61K 9/2013; A61K 9/2886; A61P 13/12; A61P 3/10

(57) ABSTRACT

The pharmaceutical composition according to the present invention contains the active ingredient dimethyl fumarate or monomethyl fumarate or a pharmaceutically acceptable salt thereof in a specific dose, and exhibits specific pharmacokinetic parameters when administered into the body. In addition, the pharmaceutical composition of the present invention has proven its efficacy and safety in type 2 diabetic nephropathy patients with albuminuria, and thus can be effectively used as a preventive or therapeutic agent for diabetic nephropathy.

### CROSS-REFERENCE TO RELATED APPLICATIONS

This is a continuation of International Application No. PCT/KR2022/004172, filed Mar. 24, 2022, which claims the benefit of Korean Patent Application No. 10-2021-0038418, filed Mar. 25, 2021, both of which are incorporated herein by reference in their entirety.

### BACKGROUND OF THE INVENTION

### 1. Field of the Invention

The present invention relates to a pharmaceutical composition containing a specific amount of dimethyl fumarate and providing a specific pharmacokinetic parameter upon administration.

### 2. Description of the Related Art

Dimethyl fumarate (DMF), the active ingredient of the present invention, is a compound represented by the following formula 1. The compound was first proposed for use in the treatment of psoriasis by a German chemist in the 1950s and has been used for the treatment of psoriasis for many years. In 1994, Fumaderm® (Fumapharm AG), a mixture of calcium, magnesium and zinc salts of dimethyl fumarate (DMF) and monoethyl fumarate (MEF), was approved in Germany for the treatment of psoriasis.

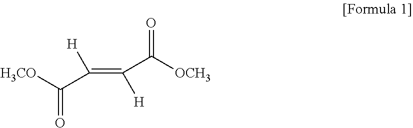

In addition to such psoriasis treatment use, U.S. Pat. No. 6,509,376 describes that a dialkyl fumarate compound to which dimethyl fumarate belongs can be used for the treatment of autoimmune diseases such as polyarthritis, multiple sclerosis, juvenile onset diabetes mellitus, systemic lupus erythematosus (sle), psoriasis, psoriasis arthritis, and neurodermatitis. In particular, U.S. Pat. No. 7,320,999 describes that dimethyl fumarate is effective for multiple sclerosis, and in March 2013, the FDA first approved it as a treatment agent for multiple sclerosis, and it is currently sold under the product name Tecfidera® in the United States and Korea. In addition, Korean Patent Publication No. 2009-0028047 describes an effect of inhibiting proliferation of vascular smooth muscle cells, and Korean Patent No. 1379427 describes that it has a preventive or therapeutic effect on renal fibrosis.

U.S. Pat. Nos. 6,355,676 and 6,509,376 describe pharmaceutical compositions in the form of enteric-coated micro-tablets or micro-pellets comprising dimethyl fumarate. International Patent Publication No. WO2010/126605 discloses a pharmaceutical composition comprising dimethyl fumarate in the form of a capsule containing enteric-coated micro-tablets. Tecfidera® currently on the market is a hard gelatin delayed-release capsule filled with micro-pellets containing dimethyl fumarate as an active ingredient.

In addition, Korean Patent No. 2197465 discloses an enteric tablet having an excellent bioavailability.

And, it is well known that dimethyl fumarate exists as monomethyl fumarate having activity in the body.

The present application was completed based on the efficacy and safety results confirmed by actually administering a pharmaceutical composition comprising dimethyl fumarate or monomethyl fumarate, an active form thereof in the body, or a pharmaceutically acceptable salt thereof as an active ingredient to a patient with type 2 diabetic nephropathy showing albuminuria.

On the other hand, diabetic nephropathy is described below.

Diabetes is increasing worldwide, and it is predicted that about 600 million people will have diabetes in 20 years. The purpose of treating diabetes, that is, controlling blood sugar, is to prevent complications caused by diabetes. Complications caused by diabetes are divided into acute complications that cause sudden loss of consciousness due to a sharp increase or decrease in blood sugar, and chronic complications that occur slowly over several years or decades without any symptoms. One of the important chronic complications is kidney disease, which is called diabetic nephropathy. Diabetic nephropathy progresses to chronic renal failure. In fact, diabetes and kidney disease are closely related so that about 40% of chronic renal failure (end-stage renal disease) patients receiving dialysis treatment in Korea and the West have diabetes as the cause. Although the frequency (prevalence) of albuminuria in adults is about 1%, it is known that the overall prevalence of albuminuria in diabetic patients is 35%. Therefore, diabetic patients clearly have a higher risk of albuminuria than the general population, and this increases as the duration of diabetes increases. Most diabetic patients develop kidney changes shortly after onset. That is, the size of the kidneys increases somewhat, and the renal blood flow and glomerular filtration rate decrease. After 10 to 15 years of being diagnosed with diabetes, about one-third of all cases develop kidney disease, which causes albuminuria. If apparent albuminuria and high blood pressure continue from this, most will progress to chronic renal failure. An important fact is that only a small percentage of diabetic patients progresses to kidney failure. It is thought that, in patients with marked hemodynamic changes in the early stage, kidney disease progresses when blood glucose control is insufficient. The reason that kidney complications do not occur in many patients may be due to genetic factors. Therefore, if there is no albuminuria 20 to 30 years after the onset of diabetes, there is little chance of developing kidney disease.

Diabetic nephropathy is initially accompanied by a decrease in glomerular filtration rate with the onset of hyperglycemia. In clinical practice, nephrologists first encounter patients with diabetic nephropathy with microalbuminuria of 30 to 300 mg/day. In the case of type 1 diabetes, overt albuminuria occurs within about 10 years in patients with microalbuminuria, and in the case of type 2 diabetes, albuminuria related to diabetic nephropathy occurs in 20 to 40% of patients.

Diabetic nephropathy is caused by hemodynamic factors and metabolic factors related to diabetes, in which various intracellular signaling pathways and various cytokines such as TGF-β (transforming growth factor-β) act. The most important means of treatment for diabetic nephropathy is RAAS (renin angiotensin aldosterone system) inhibition, and it has been demonstrated that it is excellent for the treatment of diabetic nephropathy in the protective effect of renal damage over blood pressure control. Examples of such RAAS blocking agents include ARBs (angiotensin receptor blockers), aldosterone antagonists, renin blockers, angiotensin converting enzyme 2, and the like. However, as treatment using active RAAS inhibitors rather causes side effects such as hyperkalemia and worsening of cardiovascular disease, the demand for new therapeutic agents to overcome these complications is increasing.

If it progresses to diabetic nephropathy, it is difficult to recover reversibly, so it is most important to prevent it before that. Therefore, the conventional treatment such as blood sugar and blood pressure control, dyslipidemia treatment, lifestyle improvement, and diet is very important.

Since hyperglycemia plays an important role in the development and progression of diabetic nephropathy, strict blood glucose control is important for the prevention and treatment of diabetic nephropathy. A clinical study conducted on patients with type 1 and type 2 diabetes showed that the onset of diabetic nephropathy was delayed when strict glycemic control was performed at the early stage of the disease. In a study conducted on type 1 diabetic patients, when glycemic control was strictly implemented so that the average HbA1c was about 7.2%, the incidence of micro and macroalbuminuria was reduced by 34% and 56%, respectively, compared to the control group (average HbA1c: 9.1%). In addition, after 22 years of follow-up, the risk of renal function decline (eGFR (Estimated glomerular filtration rate)<60 mL/min/1.73 m²) in the group that had previously been subjected to strict blood sugar control was reduced by more than 50% compared to the control group. This strict blood sugar lowering effect was similar to that in patients with type 2 diabetes.

The American Diabetes Association recommends that the glycemic control of diabetic patients be less than 7% HbA1c, and Korea recommends less than 6.5%. In the case of chronic renal failure, glucose production and insulin clearance in the kidneys decrease, so the risk of hypoglycemia may increase during strict glycemic control and adverse drug reactions may occur. Therefore, it is necessary to prescribe drugs and switch to insulin treatment in consideration of the patient's residual renal function.

In diabetes, blood pressure control is important to lower the risk of cardiovascular disease and inhibit the development and progression of diabetic nephropathy. According to KDIGO (kidney disease improving global outcomes) guideline, a blood pressure of 140/90 mmHg or less is recommended for people with a urine albumin excretion of 30 mg/g Cr or less as a target blood pressure to reduce cardiovascular disease-related mortality and suppress the progression of nephropathy. In addition, a lower target blood pressure of less than 130/80 mmHg is recommended for people with urinary albumin excretion greater than 30 mg/g Cr or at high risk of cardiovascular disease. In diabetic nephropathy patients with a urine albumin excretion of 30 mg/g Cr or more, ACE inhibitors and ARBs are considered primary antihypertensive agents. In patients with diabetic nephropathy accompanied by moderate to severe albuminuria 300 mg/day), these drugs showed renal protective effects by reducing albuminuria and delaying the progression of nephropathy independently of blood pressure lowering. On the other hand, in patients without albuminuria or with mild albuminuria (30˜299 mg/g Cr), these drugs did not clearly show the effect of improving renal prognosis. Although ACE inhibitors or ARBs are prescribed for the purpose of reducing albuminuria in patients with diabetic nephropathy, it is not yet clear whether they have superior effects on renal and cardiovascular prognosis compared to other antihypertensive drugs. Therefore, the use of these drugs for the purpose of preventing the occurrence of diabetic nephropathy in diabetic patients without hypertension and albuminuria is not recommended. In addition, although these drugs are considered as primary antihypertensive drugs in diabetic patients, they may exacerbate the progression of nephropathy in advanced chronic renal failure or elderly patients. Therefore, it should be prescribed while paying attention to the occurrence of side effects such as acute renal failure or hyperkalemia. In addition, treatment of dyslipidemia, lifestyle modification (diet control, quit smoking), treatment of hyperuricemia, and treatment of metabolic acidosis are considered.

Dimethyl fumarate (DMF) is bioavailable fumaric acid ester (eg, monomethyl fumarate) administered orally. It was developed as a treatment for multiple sclerosis, an immune-mediated inflammatory disease that attacks the myelin sheath of the central nervous system, and obtained marketing approval from the US FDA in March 2013 and EMA in January 2014. DMF is sold under the trade name of Tecfidera® capsules in Korea.

DMF relieves multiple sclerosis, an immune-mediated inflammatory disease that attacks the myelin sheath of the central nervous system, and prevents the invasion of cancer cells. In addition, DMF is known to decrease the proliferation of airway smooth muscle cells through the induction of heme oxygenase (HO)-1. According to a recent study, DMF was shown to increase the expression of NF-E2-related factor 2 (Nrf2). It has been proven that Nrf2 prevents diabetic nephropathy induced by streptozotocin and alleviates nephrotoxicity caused by cisplatin through the induction of antioxidant enzymes.

Renal fibrosis symptoms usually appear in patients with diabetic nephropathy, and the treatment of diabetic nephropathy entails preventing, alleviating or treating renal fibrosis symptoms.

The pathogenesis of fibrosis is generally caused by complex factors, inflammation, immunological reactions, ischemia, hemodynamic changes, and the like. Soft tissue cells damaged by the above factors activate macrophages to release a number of cytokines and growth factors. Among them TGF-β is an important factor. TGF-β activates the extracellular matrix (ECM) to generate cells and transform them into myofibroblasts. The formed fibroblasts not only increase the production of collagen, which is a key protein of ECM, but also reduce the destruction of ECM. As a result, ECM accumulates, which leads to fibrosis of the organ or tissue.

DMF inhibits the phosphorylation of Smad3 induced by TGF-β and suppresses the expression of profibrotic gene and ECM protein. In addition, DMF activates Nrf2, a transcriptional regulator important for the production of intracellular antioxidant enzymes and phase II detoxification enzymes against external oxidative stress. Nrf2 suppresses the expression of TGF-β-mediated profibrotic gene and ECM protein through ARE (antioxidant response element)-independent mechanism.

### SUMMARY OF THE INVENTION

It is an object of the present invention to provide a pharmaceutical composition comprising dimethyl fumarate or monomethyl fumarate or a pharmaceutically acceptable salt as an active ingredient and providing a pharmacokinetic parameter suitable for treating diabetic nephropathy when administered into the body.

To achieve the above object, the present invention provides a pharmaceutical composition for preventing or treating diabetic nephropathy, comprising 60 to 480 mg of dimethyl fumarate or monomethyl fumarate or a pharmaceutically acceptable salt thereof as an active ingredient, and providing one or more of the following pharmacokinetic parameters:

(a) Mean plasma monomethyl fumarate Cₘₐₓ of 1675.6 ng/mL (±10%);

(b) Mean plasma monomethyl fumarate Tₘₐₓ of 0.8 hr (±10%);

(c) Mean plasma monomethyl fumarate AUCₗₐₛₜ of 1908.0 hr·ng/mL (±10%);

(d) Mean plasma monomethyl fumarate AUC_{inf} of 1936.1 hr·ng/mL (±10%);

(e) Mean plasma monomethyl fumarate AUCₑₓₜᵣₐ of 1.4% (±10%);

(f) Mean plasma monomethyl fumarate t_{1/2} of 0.8 hr (±10%);

wherein, each of the parameters of (a) to (f) is a value when 120 mg of the active ingredient is included, and the active ingredient shows a dose-proportional linear elimination kinetics.

### Advantageous Effect

The pharmaceutical composition according to the present invention contains the active ingredient dimethyl fumarate or monomethyl fumarate or a pharmaceutically acceptable salt thereof in a specific dose, and exhibits specific pharmacokinetic parameters when administered into the body. In addition, the pharmaceutical composition of the present invention has proven its efficacy and safety in type 2 diabetic nephropathy patients with albuminuria, and thus can be effectively used as a preventive or therapeutic agent for diabetic nephropathy.

### BRIEF DESCRIPTION OF THE DRAWINGS

FIG. 1 is a graph showing the blood concentration of the enteric-coated tablet containing dimethyl fumarate of Example 11.

FIG. 2 is a graph showing the amount of change in transforming growth factor beta 1 (TGF-β1) of the test group (Example 11) compared to the baseline at the end of administration (12 weeks).

FIG. 3 is a graph showing the amount of change in transforming growth factor beta 1 (TGF-β1) of the control group (placebo) compared to the baseline at the end of administration (12 weeks).

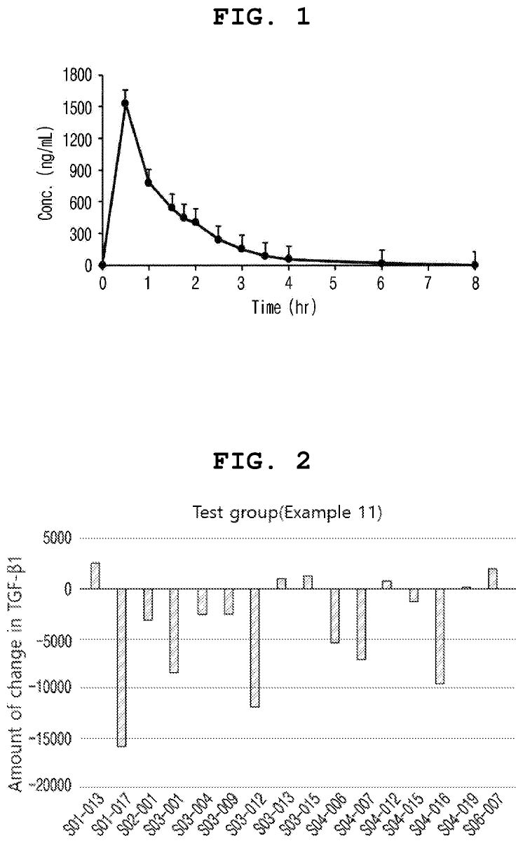

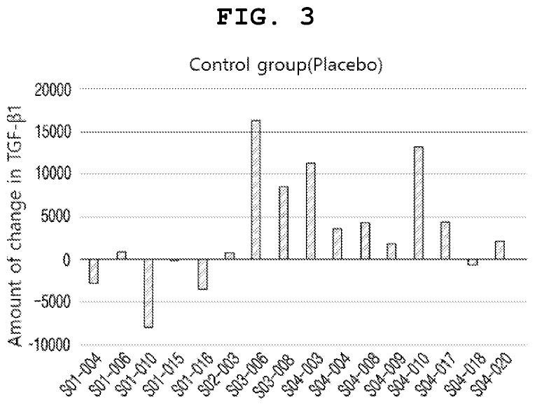

### DESCRIPTION OF THE PREFERRED EMBODIMENTS

Hereinafter, the present invention is described in detail.

The embodiments of this invention can be modified in various other forms, and the scope of the present invention is not limited to the embodiments described below. It is well understood by those in the art who has the average knowledge on this field that the embodiments of the present invention are given to explain the present invention more precisely.

In addition, the “inclusion” of an element throughout the specification does not exclude other elements, but may include other elements, unless specifically stated otherwise.

In one aspect of the present invention, the present invention provides a pharmaceutical composition for preventing or treating diabetic nephropathy, comprising 60 to 480 mg of dimethyl fumarate or monomethyl fumarate or a pharmaceutically acceptable salt thereof as an active ingredient, and providing one or more of the following pharmacokinetic parameters:

(a) Mean plasma monomethyl fumarate Cₘₐₓ of 1675.6 ng/mL (±10%);

(b) Mean plasma monomethyl fumarate Tₘₐₓ of 0.8 hr (±10%);

(c) Mean plasma monomethyl fumarate AUCₗₐₛₜ of 1908.0 hr·ng/mL (±10%);

(d) Mean plasma monomethyl fumarate AUC_{inf} of 1936.1 hr·ng/mL (±10%);

(e) Mean plasma monomethyl fumarate AUCₑₓₜᵣₐ of 1.4% (±10%);

(f) Mean plasma monomethyl fumarate t_{1/2} of 0.8 hr (±10%);

wherein, each of the parameters of (a) to (f) is a value when 120 mg of the active ingredient is included, and the active ingredient shows a dose-proportional linear elimination kinetics.

As used herein, the term “administration” refers to introduction to a patient by any suitable method, and administration can be carried out through various routes, either oral or parenteral, as long as it can reach a target tissue. Preferably, it may be oral administration. In addition, the pharmaceutical composition can be formulated in various preparations according to the desired administration mode.

For example, the pharmaceutical composition can be formulated into tablets, mini-tablets, granules, capsules, and the like.

The pharmaceutical composition contains 60 to 480 mg of dimethyl fumarate or monomethyl fumarate or a pharmaceutically acceptable salt thereof as an active ingredient, and should be administered in an amount effective to prevent or treat diabetic nephropathy.

The amount of 60 to 480 mg may correspond to an effective daily dose for preventing or treating diabetic nephropathy.

The administration method of the pharmaceutical composition can be adjusted so as to satisfy the pharmacokinetic parameter conditions described within a dose range of 60 to 480 mg per day.

The administration method is not particularly limited, but may be administered once a day or divided into two to three times and administered several times. For example, 120 mg of the active ingredient can be administered separately in the morning and afternoon, or 240 mg of the active ingredient can be administered once.

The subject to be administered can be any animal, including humans, and the animal is a mammal, such as cattle, horses, sheep, pigs, goats, camels, antelopes, dogs, and cats requiring treatment for symptoms similar to those of humans, but not always limited thereto.

The pharmaceutical composition provides one or more of the following pharmacokinetic parameters:

(a) Mean plasma monomethyl fumarate Cₘₐₓ of 1675.6 ng/mL (±10%);

(b) Mean plasma monomethyl fumarate Tₘₐₓ of 0.8 hr (±10%);

(c) Mean plasma monomethyl fumarate AUCₗₐₛₜ of 1908.0 hr·ng/mL (±10%);

(d) Mean plasma monomethyl fumarate AUC_{inf} of 1936.1 hr·ng/mL (±10%);

(e) Mean plasma monomethyl fumarate AUCₑₓₜᵣₐ of 1.4% (±10%);

(f) Mean plasma monomethyl fumarate t_{1/2} of 0.8 hr (±10%);

The Cₘₐₓ is the observed maximum drug concentration.

The Tₘₐₓ is the time to reach Cₘₐₓ, and when the maximum value occurred at two or more time points, the Tₘₐₓ was defined as the first time point.

The AUCₗₐₛₜ is the area under the concentration-time curve from the time 0 (time of administration) to the last time point (last) with measurable drug concentration, calculated by the linear trapezoidal method.

The AUC_{inf} is the area under the concentration-time curve from the time 0 (time of administration) to infinity. AUC_{inf} was calculated as the sum of AUCₗₐₛₜ plus the ratio of the last measurable drug concentration to the terminal first-order decay rate constant.

The AUCₑₓₜᵣₐ represents the percentage of AUC_{inf} at the last time point as a measurable drug concentration up to infinity calculated by (1−AUCₗₐₛₜ/AUC_{inf})×100.

The t_{1/2} is the plasma half-life calculated as 0.693/kₑ₁.

The kₑ₁ is the terminal primary decay rate constant calculated from the semi-log plot of the plasma concentration-time curve.

The pharmaceutical composition according to the present invention contains the active ingredient dimethyl fumarate or monomethyl fumarate or a pharmaceutically acceptable salt thereof in a specific dose, and exhibits specific pharmacokinetic parameters when administered into the body. The pharmaceutical composition of the present invention has proven its efficacy and safety in type 2 diabetic nephropathy patients with albuminuria, and suggests the optimal dosage of the composition, and in a more preferred aspect, the number of administration may be twice a day. The active ingredient dimethyl fumarate or monomethyl fumarate or a pharmaceutically acceptable salt thereof can be administered in a dose of 100 mg to 400 mg per one administration, in a dose of 50 mg to 400 mg, in a dose of 100 mg to 350 mg, in a dose of 100 mg to 300 mg, in a dose of 100 mg to 250 mg, in a dose of 100 mg to 150 mg, in a dose of 200 mg to 250 mg, in a dose of 330 mg to 400 mg, in a dose of 330 mg to 480 mg, in a dose of 50 mg to 100 mg, in a dose of about 60 mg, in a dose of about 120 mg, in a dose of about 240 mg, in a dose of about 360 mg, or in a dose of about 480 mg. Preferably, it can be administered in a dose of 110 to 250 mg, and most preferably, it can be administered in a dose of 115 mg to 125 mg. For reference, dimethyl fumarate or monomethyl fumarate or a pharmaceutically acceptable salt thereof has demonstrated dose-proportional linear elimination kinetics from 120 mg to 360 mg.

The pharmaceutical composition can be provided in various formulations, and as a specific example, can be provided in the form of an enteric-coated tablet. More specifically, it can be provided as an enteric-coated tablet disclosed in Korean Patent No. 2197465.

The enteric-coated tablet disclosed in Korean Patent No. 2197465 includes a core comprising dimethyl fumarate or monomethyl fumarate or a pharmaceutically acceptable salt thereof as an active ingredient; an enteric coating layer; and a seal coating layer comprising a cellulose-based polymer between the core and the enteric coating layer.

The enteric coating layer is included in an amount of 6 to 9 weight part based on 100 weight part of the core, and the seal coating layer is included in an amount of 1 to 3 weight part based on 100 weight part of the core.

The particle size distribution of the dimethyl fumarate or monomethyl fumarate or a pharmaceutically acceptable salt thereof satisfies one or more of the following conditions:

(a) The mean particle size of the lower 90% of the particles (D90) is 100 μm or less;

(b) The mean particle size of the lower 50% of the particles (D50) is 50 μm or less; and

(c) The mean particle size of the lower 10% of the particles (D10) is 20 μm or less.

In the case of commercially available capsule formulations, loss of dimethyl fumarate may occur during the manufacturing process, and there are problems such as impossible administration to a patient group, where taking animal-derived ingredients is contraindicated due to religious problems, and convenience in taking. On the other hand, in the case of the enteric-coated tablet, by controlling the content of the enteric coating layer, dimethyl fumarate is stably delivered to the absorption site and rapidly dissipated, resulting in a therapeutic effect. The enteric coating layer is typically used in an amount of 10 to 12 weight % or 10 to 13 weight % based on the total weight of the tablet core. However, in the present application, by using 6 to 9 weight part of dimethyl fumarate or monomethyl fumarate or a pharmaceutically acceptable salt thereof based on 100 weight part of the tablet core, dissolution proceeds rapidly at the absorption site, thereby ensuring excellent bioavailability.

wherein, the active ingredient can be included in an amount of 20 to 60 weight % based on the core, can be included in an amount of 25 to 55 weight %, can be included in an amount of 30 to 50 weight %, can be included in an amount of 40 to 45 weight %, can be included in an amount of 43 to 45 weight %, or can be included in an amount of about 44 weight %.

The core can include one or more pharmaceutically acceptable additives selected from the group consisting of excipients, disintegrants and lubricants. In this case, the excipient can be included in an amount of 30 to 45 weight %, the disintegrant can be included in an amount of 10 to 20 weight %, and the lubricant can be included in an amount of 0.1 to 2 weight % based on the core. The pharmaceutically acceptable additives are not limited to the excipients, disintegrants, and lubricants, and may be used as long as they are commonly used pharmaceutically additives. For example, additives such as excipients, binders, disintegrants, antioxidants, surfactants, lubricants, plasticizers, and pigments can be included.

Examples of the excipient include starch, lactose, lactose anhydrous, microcrystalline cellulose, silicified microcrystalline cellulose, hypromellose, silicic anhydride, calcium phosphate, anhydrous calcium phosphate, calcium hydrogen phosphate, anhydrous calcium hydrogen phosphate, calcium silicate, dextrin, dextrose, dextrate, mannitol, maltose, sorbitol, sucrose, polyethylene glycol, sodium chloride, and the like, and these can be used alone or in combination of two or more. Preferably, silicified microcrystalline cellulose can be used as the excipient.

Examples of the disintegrant include crospovidone, croscarmellose sodium, sodium glycolate starch, pregelatinized starch, low-substituted hydroxypropyl cellulose, grain starch, and the like, and these can be used alone or in combination of two or more. Preferably, croscarmellose sodium can be used as the disintegrant.

Examples of the lubricant include magnesium stearate, stearic acid, talc, silicon dioxide, colloidal silicon dioxide, sodium stearyl fumarate, sodium lauryl sulfate, poloxamer, and the like, and these can be used alone or in combination of two or more. Preferably, colloidal silicon dioxide or magnesium stearate can be used, and most preferably, colloidal silicon dioxide and magnesium stearate can be used as the lubricant.

Examples of the plasticizer include triethyl citrate, acetyltributyl citrate, glycerol acetic acid fatty acid ester, triacetin, dibutylphthalate, polysorbate 80, polyethylene glycol, propylene glycol, and the like, and these can be used alone or in combination of two or more.

Examples of the binder include povidone, copovidone, methyl cellulose, hydroxymethyl cellulose, hydroxypropyl methyl cellulose, hydroxypropyl cellulose, hydroxyethyl cellulose, gelatin, guar gum, xanthan gum, and the like, and these can be used alone or in combination of two or more.

Examples of the antioxidant include dibutylhydroxytoluene, butylhydroxytoluene, butylhydroxyanisole, tert-butylhydroquinone, propyl gallate, vitamin C, and the like, and these can be used alone or in combination of two or more.

Examples of the surfactant include sodium lauryl sulfate, sodium stearate, polysorbate 80, poloxamer, and the like, and these can be used alone or in combination of two or more.

A seal coating layer can be further included between the core and the enteric coating layer. In this case, the seal coating layer may be referred to as an intermediate coating layer, a primary coating layer, or a non-enteric coating layer. The seal coating layer can include a cellulose-based polymer, preferably hydroxypropylmethylcellulose, but not always limited thereto. The cellulose-based polymer is not particularly limited as long as it is a non-enteric coating base, and it can be at least one selected from the group consisting of polyvinyl alcohol (PVA), polyethylene glycol, polyvinyl alcohol-polyethylene glycol graft copolymer (eg, Kollicoat-IR), ethyl cellulose, hydroxypropyl cellulose (HPC), lactose and mannitol.

The seal coating layer can be included in an amount of 1 to 3 weight part, can be included in 1 to 2 weight part, can be included in about 1.5 weight part, or can be included in about 2 weight part based on 100 weight part of the core.

The core can further include an alkalizing agent, wherein, the weight ratio of the active ingredient and the alkalizing agent can be 12:0.5 to 12:2, 12:0.7 to 12:1.8, 12:0.8 to 12:1.5, 12:0.9 to 12:1.3, 12:0.9 to 12:1.1, and preferably can be 2:1.

The alkalizing agent can be included in an amount of 2 to 5 weight %, 2.5 to 4.5 weight %, 3 to 4 weight %, 3.5 to 4 weight %, and about 3.7 weight % based on the core.

As the alkalinizing agent, a known alkalizing agent can be used to increase the aqueous solubility of the active ingredient, and preferably meglumine or a pharmaceutically acceptable salt thereof can be used as the alkalizing agent to improve the compressibility, adsorption, disintegration, stability, and the like suitable for tablets.

As the enteric coating layer, one or more enteric coating polymers selected from the group consisting of an enteric acrylic acid-based copolymer selected from the group consisting of styrene-acrylic acid copolymer, ethyl methacrylate methacrylate copolymer, methyl acrylate methacrylate octyl acrylate copolymer, and ethyl methacrylate acrylate copolymer; an enteric cellulose-based polymer selected from the group consisting of hydroxypropylmethylcellulose acetate succinate, hydroxypropylmethylcellulose phthalate, hydroxymethylethylcellulose phthalate, cellulose acetate phthalate, cellulose acetate maleate, cellulose acetate succinate, cellulose acetate maleate, cellulose benzoate phthalate, cellulose propionate phthalate, methylcellulose phthalate, carboxymethylethylcellulose, ethylhydroxyethylcellulose phthalate, carboxymethylethylcellulose and ethylhydroxyethylcellulose phthalate; an enteric maleic acid-based copolymer selected from the group consisting of vinyl acetate maleic acid anhydride copolymer, styrene maleic acid anhydride copolymer, styrene maleic acid monoesterol copolymer, vinylmethyl ether maleic acid anhydride copolymer, ethylene maleic acid anhydride copolymer, vinyl butyl ether maleic acid anhydride copolymer, acrylonitrile methyl acrylate maleic acid anhydride copolymer and butyl acrylate styrene maleic acid anhydride copolymer; and an enteric polyvinyl-based polymer selected from the group consisting of polyvinyl alcohol phthalate, polyvinyl acetacetal phthalate, polyvinyl butyrate phthalate and polyvinyl acetacetal phthalate; can be used, but if it is a pharmaceutically acceptable enteric coating base, it is not particularly limited. The enteric tablet according to the present invention can solve the difference in quality between batches due to non-uniformity of mixing that may occur when mixing two or more types of coating bases by mixing additives other than the enteric coating base.

The enteric coating layer can be formed using an enteric coating base comprising the enteric coating polymer in an amount of 20 to 80 weight %, wherein the polymer included in the enteric coating base can be included in an amount of 20 to 60 weight %, can be included in an amount of 40 to 80 weight %, can be included in an amount of 40 to 60 weight %, can be included in an amount of 35 to 45 weight %, can be included in an amount of 55 to 65 weight %, or can be included in an amount of about 60 weight %.

If the enteric coating layer is 5 weight part or less based on 100 weight part of the core, there may be a problem that the drug is eluted and decomposed in the stomach. If the enteric coating layer is 9 weight part or more based on 100 weight part of the core, the absorption rate of the drug in the body is lowered, and it takes a long time to reach the effective concentration, so that the therapeutic effect is not properly exhibited. The content range of the enteric coating layer according to the present application is preferable to control the dissolution rate so that the salt is stably delivered to the absorption site in the body and dissolution is possible to sufficiently exhibit the therapeutic effect.

The particle size distribution of the dimethyl fumarate or monomethyl fumarate or a pharmaceutically acceptable salt thereof satisfies one or more of the following conditions: (a) The mean particle size of the lower 90% of the particles (D90) is 100 μm or less; (b) The mean particle size of the lower 50% of the particles (D50) is 50 μm or less; and (c) The mean particle size of the lower 10% of the particles (D10) is 20 μm or less. In addition, the particle size distribution satisfies one or more of the following conditions: (a) The mean particle size of the lower 90% of the particles (D90) is 80 μm or less; (b) The mean particle size of the lower 50% of the particles (D50) is 40 μm or less; and (c) The mean particle size of the lower 10% of the particles (D10) is 15 μm or less, or (a) The mean particle size of the lower 90% of the particles (D90) is 50 μm or less; (b) The mean particle size of the lower 50% of the particles (D50) is 30 μm or less; and (c) The mean particle size of the lower 10% of the particles (D10) is 10 μm or less.

The thickness of the coating layer of the enteric-coated tablet can be 20 μm to 90 μm, can be 30 μm to 80 μm, can be 30 μm to 50 μm, can be 60 μm to 80 μm, can be 35 μm to 50 μm, can be 65 μm to 80 μm, can be 35 μm to 80 μm, or can be 40 μm to 75 μm. In this case, the coating layer of the enteric-coated tablet may be the thickness of the enteric coating layer, or the thickness of the coating layer including the seal coating layer and the enteric coating layer.

The enteric-coated tablet can be prepared by the conventional method for preparing tablets such as dry/wet granulation method, direct powder compression method, or direct compression method, and preferably can be prepared by direct compression method.

The enteric-coated tablet can be in the form of a powder, and is preferably prepared as an enteric-coated tablet in solid form, but it is not impossible to manufacture in the form of a liquid form, and this is not excluded from the scope of a right of the present invention.

In another aspect of the present invention, the pharmaceutical composition may be provided for preventing, alleviating or treating organ fibrosis.

wherein, the organ fibrosis is at least one selected from the group consisting of renal fibrosis, cardiac fibrosis, pancreatic fibrosis, lung fibrosis, vascular fibrosis, bone marrow fibrosis, liver fibrosis, scleroderma, cystic fibrosis and intestinal fibrosis;

The renal fibrosis is at least one selected from the group consisting of renal failure, diabetic nephropathy, glomerulosclerosis, renal tubular fibrosis, glomerulonephritis, chronic renal failure, acute kidney injury, chronic kidney disease, end-stage renal disease and albuminuria;

The liver fibrosis is at least one selected from the group consisting of cirrhosis, hepatic nephrotic syndrome, peliosis hepatis, metabolic liver disease, chronic liver disease, hepatitis B virus infection, hepatitis C virus infection, hepatitis D virus infection, schistosomiasis, alcoholic liver disease, non-alcoholic steatohepatitis, obesity, diabetes, protein deficiency, coronary artery disease, autoimmune hepatitis, cystic fibrosis, alpha-1 antitrypsin deficiency and primary biliary cirrhosis;

The lung fibrosis is at least one selected from the group consisting of bronchitis, acute bronchitis, diffuse panbronchitis (DPB), bronchiolitis, idiopathic pulmonary fibrosis (IPF), acute interstitial pneumonia, lung transplantation, radiation-induced pulmonary fibrosis, acute respiratory syndrome (ARDS), chronic obstructive pulmonary disease (COPD), asthma, bronchiectasis, pulmonary tuberculosis, pneumonia, pneumoconiosis, hypersensitivity pneumonitis, pulmonary edema and sarcoidosis.

The present invention also provides a method for preventing or treating diabetic nephropathy, comprising a step of administering a pharmaceutical composition containing 60 to 480 mg of dimethyl fumarate or monomethyl fumarate or a pharmaceutically acceptable salt thereof as an active ingredient, and the administration of the pharmaceutical composition to a subject provides one or more of the following pharmacokinetic parameters:

(a) Mean plasma monomethyl fumarate Cₘₐₓ of 1675.6 ng/mL (±10%);

(b) Mean plasma monomethyl fumarate Tₘₐₓ of 0.8 hr (±10%);

(c) Mean plasma monomethyl fumarate AUCₗₐₛₜ of 1908.0 hr·ng/mL (±10%);

(d) Mean plasma monomethyl fumarate AUC_{inf} of 1936.1 hr·ng/mL (±10%);

(e) Mean plasma monomethyl fumarate AUCₑₓₜᵣₐ of 1.4% (±10%);

(f) Mean plasma monomethyl fumarate t_{1/2} of 0.8 hr (±10%);

wherein, each of the parameters of (a) to (f) is a value when 120 mg of the active ingredient is included, and the active ingredient shows a dose-proportional linear elimination kinetics.

The present invention also provides a use of a pharmaceutical composition containing 60 to 480 mg of dimethyl fumarate or monomethyl fumarate or a pharmaceutically acceptable salt thereof as an active ingredient in the manufacture of a medicament for the prevention or treatment of diabetic nephropathy, and the administration of the pharmaceutical composition to a subject provides one or more of the following pharmacokinetic parameters:

(a) Mean plasma monomethyl fumarate Cₘₐₓ of 1675.6 ng/mL (±10%);

(b) Mean plasma monomethyl fumarate Tₘₐₓ of 0.8 hr (±10%);

(c) Mean plasma monomethyl fumarate AUCₗₐₛₜ of 1908.0 hr·ng/mL (±10%);

(d) Mean plasma monomethyl fumarate AUC_{inf} of 1936.1 hr·ng/mL (±10%);

(e) Mean plasma monomethyl fumarate AUCₑₓₜᵣₐ of 1.4% (±10%);

(f) Mean plasma monomethyl fumarate t_{1/2} of 0.8 hr (±10%);

wherein, each of the parameters of (a) to (f) is a value when 120 mg of the active ingredient is included, and the active ingredient shows a dose-proportional linear elimination kinetics.

It is obvious that the contents described for the above-mentioned pharmaceutical composition can be applied as they are within the range that can achieve the object of the invention in relation to the treatment method or use.

On the other hand, the patient set as the subject of the clinical trial in this specification is a type 2 diabetic nephropathy patient, but the experimental results derived from the clinical trial are not limited to the medicinal use for diabetic nephropathy, and can also be used for the treatment of fibrosis of all organs.

In this application, the active ingredient content, administration method/dose, and pharmacokinetic parameters at the time of administration of the pharmaceutical composition containing DMF that can ensure excellent efficacy and safety in the treatment of diabetic nephropathy were proved through advanced clinical trials.

In this regard, the pathogenesis of fibrosis is generally caused by complex factors, inflammation, immunological reactions, ischemia, hemodynamic changes, and the like. Soft tissue cells damaged by the above factors activate macrophages to release a number of cytokines and growth factors. Among them TGF-β is an important factor. TGF-β activates the extracellular matrix (ECM) to generate cells and transform them into myofibroblasts. The formed fibroblasts not only increase the production of collagen, which is a key protein of ECM, but also reduce the destruction of ECM. As a result, ECM accumulates, which leads to fibrosis of the organ or tissue.

DMF inhibits the phosphorylation of Smad3 induced by TGF-β and suppresses the expression of profibrotic gene and ECM protein. In addition, DMF activates Nrf2, a transcriptional regulator important for the production of intracellular antioxidant enzymes and phase II detoxification enzymes against external oxidative stress. Nrf2 suppresses the expression of TGF-β-mediated profibrotic gene and ECM protein through ARE (antioxidant response element)-independent mechanism. The results of conventional efficacy tests in this regard are summarized below.

A. In Vitro Test

(Oh et al., Dimethylfumarate Attenuates Renal Fibrosis via NF-E2-Related Factor 2-Mediated Inhibition of Transforming Growth Factor-β/Smad Signaling, PLoS One. 2012; 7(10):e45870)

\textcircled{1} Inhibition of PAI-1, a-SMA, Fibronectin and Collagen Type 1 Increased by TGF-β

The effect of DMF on the TGF-β-mediated expression of profibrotic gene and ECM protein gene in a rat renal fibroblast cell line (NRK-49F) was evaluated. DMF inhibited the TGF-β-mediated expression of PAI-1, a-SMA, and fibronectin mRNA and protein dose-dependently. The expression of collagen type 1 mRNA and protein was not increased after 6 hours of the treatment of TGF-β, but the expression was induced after 24 hours of the treatment, and then DMF was shown to dose-dependently inhibit collagen type 1 induced by TGF-β. The inhibitory effect of DMF on the TGF-β-induced expression of PAI-1 protein was additionally confirmed in RMCs and rat glomerular mesangial cells. As a result, DMF suppressed the TGF-β-induced expression of profibrotic gene and ECM protein in rat kidney cell lines.

\textcircled{2} Inhibition of TGF-β/Smad3 Signaling Pathway by DMF

To confirm that DMF suppresses the expression of profibrotic gene and ECM protein through the inhibition of TGF-β-induced Smad signaling, the effect of DMF on the structure of a luciferase reporter carrying the PAI-1 promoter (PAI-1-Luc) containing three binding sites for Smad3 was evaluated. In addition, the effect of dimethyl fumarate on the activity of the (CAGA)₉MLP-Luc promoter, a reporter structure containing 9 copies of the Smad3 binding site, was evaluated. Dimethyl fumarate inhibited the activity of the PAI-1 promoter induced by TGF-β in AD-293 cells, derivatives of HEK-293 human embryonic kidney cell line. In addition, dimethyl fumarate inhibited the activity of the (CAGA)₉MLP-Luc promoter induced by TGF-β or the constitutively active TGF-β type 1 receptor (ALK5). Since the phosphorylation of Smad3 is an important step to achieve TGF-β signaling, the effect of DMF on the phosphorylation of Smad3 (p-Smad3) was evaluated. The treatment of DMF inhibited the Smad3 phosphorylation induced by TGF-β in NRK-49F and RMC cells. These results show that DMF negatively affects the TGF-β-mediated transcription through the inhibition of the phosphorylation of Smad3.

\textcircled{3} Nrf2 Increase, Inhibition of Expression of Nrf2 PAI-1, a-SMA, Fibronectin and Collagen Type 1

DMF is known as an activator of the transcription factor Nrf2 involved in the pathogenesis of renal fibrosis. Therefore, it was tested whether Nrf2 affects the inhibitory effect of DMF on the TGF-β-induced expression of profibrotic gene and ECM protein. As a result, the treatment of DMF resulted in a sharp increase in the Nrf2 protein expression. The expression of Nrf2 in NRK-49F cells was decreased gradually from 1 hour after the DMF treatment, but the expression of Nrf2 was increased until 24 hours after the simultaneous treatment with DMF and TGF-β. In addition, DMF induced the nuclear accumulation of Nrf2 dose-dependently. As the p62-mediated stabilization of Nrf2 was recently proposed as a mechanism distinct from antioxidants for the Nrf2 activation, the effect of DMF on p62 in the DMF-induced expression of Nrf2 was evaluated. Contrary to the rapid induction of Nrf2 by DMF, the expression of p62 was increased 6 h after the DMF treatment and up to 12 hours thereafter. These results showed that DMF increased the expression of Nrf2 by a mechanism distinct from p62.

In addition, in order to confirm the decrease in the expression of ECM during Nrf2 overexpression, the effect on the expression of ECM including collagen type 1 was confirmed at the RNA and protein levels by overexpressing Nrf2 using adenovirus (Ad-Nrf2). As a result, Nrf2 (Ad-Nrf2) overexpressed by adenovirus reduced the expression of PA1-1, a-SMA and fibronectin mRNA and protein in NRK-49F cells. In addition, Ad-Nrf2 reduced the expression of collagen type 1 mRNA 24 hours after the TGF-β treatment. Additionally, it was confirmed that DMF rapidly increased the expression of Nrf2 protein in RMC cells, and that Ad-Nrf2 suppressed the TGF-β-induced expression of ECM in RMC cells. In addition, Nrf2 transfection dose-dependently reduced the activity of the PAI-1 promoter induced by TGF-β or ALK5 co-transfection. In contrast, the promoter activity of NQO1, a target gene of Nrf2, was remarkably activated by Nrf2 transfection. In conclusion, it was confirmed that Nrf2 activated by DMF suppressed the TGF-β-induced expression of profibrotic genes.

4̅ Inhibition of TGF-β/Smad3 Signaling Pathway by Nrf2

To confirm the inhibitory effect of Nrf2 on TGF-β/Smad signaling, the effect of Ad-Nrf2 on the TGF-β-induced Smad3 phosphorylation was evaluated. As a result, Ad-Nrf2 inhibited the TGF-β-induced Smad3 phosphorylation, but had no effect on the total Smad3 and Smad4 protein expression in NRK-49F cells and RMC cells. To further confirm that Nrf2 affects the inhibition of the TGF-β/Smad3 and ECM protein expression due to DMF, the expression of endogenous Nrf2 was lowered by transfecting AD-293 cells with siRNA (small interfering RNA) for Nrf2. As a result, Nrf2-siRNA successfully inhibited the expression of Nrf2 and significantly blocked the inhibitory effect of DMF on the TGF-β-mediated (CAGA)₉MLP-Luc promoter activity. In addition, Nrf2-siRNA reversed the inhibitory effect of DMF on the expression of collagen type 1 induced by TGF-β-. These results indicate that Nrf2 mediates the inhibitory effect of DMF on the TGF-β/Smad signaling pathway and TGF-β-induced ECM protein expression.

\textcircled{5} Association of Antioxidant Enzymes NQO1 and HO-1 with TGF-β/Smad Signaling Pathway

While Nrf2 target genes such as NQO1 and HO-1 prevent renal fibrosis caused by reactive oxygen species (ROS), it has been demonstrated that ROS mediates renal fibrosis induced by TGF-β. Accordingly, the expression levels of the activating enzymes HO-1 (heme-oxygenase-1) and NQO1 according to DMF were confirmed in NRK-49F cells. Cells were treated with DMF (40 μmol/1) and semi-quantitative RT-PCR analysis was performed. As a result, DMF increased the expression of NQO1 and HO-1 in NRK-49F cells. To confirm whether the induction of NQO1 and HO-1 is necessary for the inhibitory effect of DMF on the TGF-β/Smad signaling pathway, the expression of NQO-1 and HO-1 induced by DMF was knocked down with NQO1 and HO-1 siRNA. As a result, the activity of the (CAGA)₉MLP-luc promoter decreased by DMF was not restored by these siRNAs. In addition, ES936 or SnPP, the chemical inhibitor of NQO1 or HO-1, did not reverse the inhibitory effect of DMF on the TGF-β/Smad signaling and ECM expression. Further, it was confirmed that there was no difference in the expression of PAI-1, α-SMA, and fibronectin when the control-siRNA, NQO1-siRNA and HO-1-siRNA treated groups were compared. From the above results, it was confirmed that the expression of ECM decreased by DMF was not related to these antioxidant enzymes.

B. In Vivo Test

Efficacy of Nrf2 Activator DMF in Unilateral Ureteral Obstruction (UUO)-Induced Renal Fibrosis Model

The efficacy of DMF, an Nrf2 activator, was evaluated in a UUO-induced renal fibrosis model. As a result, it was confirmed that DMF, an Nrf2 activator, decreased the expression of p-Smad3, which is a renal fibrosis increased by UUO, and decreased the expression of collagen type I, fibronectin, PAI-1 and α-SMA related to fibrosis. 25 mg/kg and 50 mg/kg of DMF showed similar reduction effects, so 25 mg/kg is considered appropriate in this animal model.

In summary, the enteric tablet according to the present invention contains the active ingredient dimethyl fumarate or monomethyl fumarate or a pharmaceutically acceptable salt thereof in a specific dose, and the efficacy and safety of the enteric tablet of the present invention have been proven in type 2 diabetic nephropathy patients with albuminuria upon administration, so that it can be effectively used as a preventive or therapeutic agent for diabetic nephropathy and organ fibrosis. The above content is supported by Examples and Experimental Examples to be described later.

Hereinafter, the present invention will be described in detail by the following examples and experimental examples. However, the following examples and experimental examples are only for illustrating the present invention, and the contents of the present invention are not limited thereto.

### <Example> Preparation of Enteric Coated Tablets

Each composition constituting the enteric-coated tablet is shown in Tables 1 and 2 below.

TABLE 1

| | | | Amount (mg/tablet) | | | | | | |
|---|---|---|---|---|---|---|---|---|---|
| | | | Example | Example | Example | Example | Example | Example | Example |
| | | Component | 1 | 2 | 3 | 4 | 5 | 6 | 7 |
| Core | Main | Dimethyl | 120.0 | 120.0 | 120.0 | 120.0 | 120.0 | 120.0 | 120.0 |
| | component | fumarate | | | | | | | |
| | Alkalizing | Meglumine | 10.0 | 10.0 | 10.0 | 10.0 | 10.0 | 10.0 | — |
| | agent | | | | | | | | |

| | | | | | | | |
|---|---|---|---|---|---|---|---|
| Excipient (silica | 140.0 | 140.0 | 140.0 | 140.0 | 140.0 | 140.0 | 140.0 |
| microcrystalline | | | | | | | |
| cellulose), disintegrant | | | | | | | |
| (croscarmellose | | | | | | | |
| sodium and/or | | | | | | | |
| crospovidone), | | | | | | | |
| lubricant (colloidal | | | | | | | |
| silicon dioxide and/or | | | | | | | |
| magnesium stearate) | | | | | | | |

| Naked tablet | | 270.0 | 270.0 | 270.0 | 270.0 | 270.0 | 270.0 | 260.0 |
|---|---|---|---|---|---|---|---|---|
| Primary | OPADRY | 5.4 | 5.4 | 5.4 | 5.4 | 5.4 | 5.4 | 5.2 |
| coating | 03K19229 | | | | | | | |
| Secondary | ACRYL- | 10.8 | 16.2 | 21.6 | — | — | — | — |
| coating | EZE MP | | | | | | | |
| | 93018508 | | | | | | | |
| | ACRYL- | — | — | — | 16.2 | 21.6 | 21.6 | 20.8 |
| | EZE MP | | | | | | | |
| | 93018509 | | | | | | | |

TABLE 2

| | | | Amount (mg/tablet) | | | | |
|---|---|---|---|---|---|---|---|
| | | Component | Example 8 | Example 9 | Example 10 | Example 11 | Example 12 |
| Core | Main | Dimethyl | 240.0 | 240.0 | 240.0 | 120.0 | 120.0 |
| | component | fumarate | | | | | |
| | Alkalizing | Meglumine | 20.0 | — | — | — | — |
| | agent | | | | | | |

| | | | | | |
|---|---|---|---|---|---|
| excipient (silica | 280.0 | 300.0 | 300.0 | 150.0 | 150.0 |
| microcrystalline | | | | | |
| cellulose), disintegrant | | | | | |
| (croscarmellose | | | | | |
| sodium and/or | | | | | |
| crospovidone), | | | | | |
| lubricant (colloidal | | | | | |
| silicon dioxide and/or | | | | | |
| magnesium stearate) | | | | | |

| Naked tablet | | 540.0 | 540.0 | 540.0 | 270.0 | 270.0 |
|---|---|---|---|---|---|---|
| Primary coating | OPADRY | 10.8 | 8.0 | 8.0 | 4.0 | 4.0 |
| | 03K19229 | | | | | |
| Secondary coating | ACRYL- | — | 43.2 | 54.0 | 16.2 | 22.0 |
| | EZE MP | | | | | |
| | 93018508 | | | | | |
| | ACRYL- | 43.2 | — | — | — | — |
| | EZE MP | | | | | |
| | 93018509 | | | | | |

Preparation of Enteric-Coated Tablets Containing Dimethyl Fumarate

The angle of repose of the mixture containing dimethyl fumarate is 40° or less, and it is usually evaluated that the fluidity is good enough to allow direct tableting if the angle of repose is less than 40°. On the other hand, when the wet granulation method is applied to improve fluidity, there is concern about loss due to sublimation of dimethyl fumarate by the use of solvent and drying. Therefore, enteric-coated tablets containing dimethyl fumarate were prepared as follows by minimizing contact with moisture and applying a direct compression method with a simple manufacturing process.

The enteric-coated tablets according to Examples 1 to 12 were prepared through the following step: preparing a core (naked tablet, that is, a tablet in a compressed state without coating) by mixing dimethyl fumarate and pharmaceutically acceptable additives (excipient (silica microcrystalline cellulose), disintegrant (croscarmellose sodium and/or crospovidone), lubricant (colloidal silicon dioxide and/or magnesium stearate), alkalizing agent (meglumine)) according to the compositions of tables 1 and 2, and tableting the mixture; primary coating (seal coating) the core with a coating solution in which a non-enteric coating base is dissolved in a solvent; and secondary coating the core with a coating solution in which an enteric coating base is dissolved in a solvent.

When a seal coating (primary coating) is performed before enteric coating, there is an advantage in that adhesion of the enteric coating base to the tablet surface can be increased and acid resistance can be increased. wherein, a polyvinyl alcohol (PVA) base can be used as the seal coating base, but when using the PVA base, the polymer ratio in the coating base is low, so it should be coated with about 6 to 10% of the weight of the naked tablet thicker than the hydroxypropylmethylcellulose (HPMC) base. In addition, depending on the surface and curvature of the tablet, the enteric-based coating is not uniformly applied, so there is a high possibility that acid resistance is impaired. The PVA base should be coated with water-based coating using water as a solvent and dried for a long time at a high temperature of 45° C. or higher. However, water-based coating is not an appropriate coating method because dimethyl fumarate is unstable to moisture and has a property of sublimation at high temperature, which may cause loss.

On the other hand, when seal-coating is performed with the hydroxypropyl methylcellulose (HPMC) base, thin coating is possible with a coating ratio of about 1.5 to 3% of the weight of the naked tablet, oil-based coating using ethanol organic solvent is possible, and short-drying at a low temperature of about 25-35° C. is possible, so that the loss of dimethyl fumarate can be minimized. In addition, when the HPMC base is used, there is an advantage in that the enteric coating film is stably maintained while the enteric coating base containing the copolymer is well attached to the surface of the seal coating film. Therefore, the enteric-coated tablet containing dimethyl fumarate according to Example used OPADRY 03K19229 mainly composed of HPMC as a seal coating base.

ACRYL-EZE MP, the enteric coating base, is classified into ACRYL-EZE MP 93O18508 and ACRYL-EZE MP 93O18509 according to the composition ratio of the metharcylic acid and ethyl acrylate copolymer copolymer. Also, as shown in table 3 below, ACRYL-EZE MP is classified as ACRYL-EZE MP 93O18508 when the weight ratio of methacrylate ethyl acrylate is 60 w/w %, and as ACRYL-EZE MP 93O18509 when the weight ratio of methacrylate ethyl acrylate is 40 w/w %. As the enteric coating base, there is also a hydroxypropyl methylcellulose phthalate-based coating base in addition to the methacrylate ethyl acrylate copolymer. Since the hydroxypropyl methylcellulose phthalate-based coating base uses a large amount of organic solvent, the possibility of detecting residual solvent is high, and the coating time is also longer than that of the methacrylate ethyl acrylate copolymer-based coating base, so it may not be generally suitable for use.

TABLE 3

| Component | w/w % | Brand |
|---|---|---|
| Methacrylate | 60 | ACRYL-EZE MP 93018508 |
| ethylacrylate | | |
| copolymer | 40 | ACRYL-EZE MP 93018509 |

### <Experimental Example 1> Evaluation of Dissolution Rate According to Particle Size of Dimethyl Fumarate

In the case of poorly soluble drugs, the solubility of the drug tends to improve as the degree of solubilization increases as the particle size of the drug decreases according to “Noyes-Whitney equation”. Therefore, the particle size of dimethyl fumarate was adjusted under the conditions shown in table 4 below, and the comparative dissolution patterns of the tablet of Example 11 containing pulverized dimethyl fumarate (that is, dimethyl fumarate in a finely pulverized state to D90 100 μm or less) and the tablet of Example 6 containing non-pulverized dimethyl fumarate were evaluated at pH 6.8. To evaluate the dissolution rate, a buffer solution of pH 6.8 (Mcilvane buffer) was prepared and a dissolution test was performed on each eluate according to the second method (paddle method). Specifically, the buffer solution was 900 mL, the stirring speed was 75 rpm, and the temperature of the buffer solution was maintained at 37±0.5° C. After the start of the dissolution test, the final time point was set based on the general time to stay in the internal organ (intestine) representing the pH 6.8, and the intermediate time points were set at regular intervals to collect the sample solution, filtered through a filter, and analyzed by high performance liquid chromatography (HPLC). The results are shown in table 5 below.

TABLE 4

| | D10 | D50 | D90 |
|---|---|---|---|
| Example 11 | 20 μm or less | 50 μm or less | 100 μm or less |
| Example 6 | 20 μm or more | 50 μm or more | 100 μm or more |

TABLE 5
Dissolution rate (%)

| Min. | Example 11 | Example 6 |
|---|---|---|
| 0 | 0.0 | 0.0 |
| 5 | 83.3 | 16.0 |
| 10 | 93.8 | 30.9 |
| 15 | 94.9 | 44.4 |
| 30 | 95.1 | 69.1 |
| 45 | 94.8 | 79.6 |
| 60 | 94.6 | 84.5 |
| 90 | 93.5 | 88.2 |
| 120 | 92.2 | 88.9 |

As a result, it was confirmed that the dissolution rate of the enteric-coated tablet containing dimethyl fumarate in the pH 6.8 solution was significantly affected by the particle size from the initial to the intermediate time point.

Specifically, when D90 was greater than 100 μm (Example 6), the dissolution rate was significantly decreased. Therefore, it is preferable that the average particle size of the lower 90% of the dimethyl fumarate particles (D90) be 100 μm or less for the initial rapid drug release.

### <Experimental Example 2> Phase 2a Clinical Trial to Evaluate Safety and Efficacy of Example 11 Tablet in Diabetic Nephropathy Patients

A multicenter, randomized, double-blind, placebo-controlled, parallel-designed, phase 2a study to evaluate the efficacy and safety of administering the enteric tablet of Example 11 for 12 weeks in type 2 diabetic nephropathy patients with albuminuria

A clinical trial evaluation was conducted to derive the optimal dosage of the enteric-coated tablet containing dimethyl fumarate prepared in example above. The specific evaluation target drug was the enteric tablet of Example 11.

1. Clinical Trial Design Contents

1-1. Clinical Trial Design

This clinical trial is designed to evaluate the efficacy, safety and pharmacokinetic properties of administering the enteric tablet of Example 11 for 12 weeks in type 2 diabetic nephropathy patients with albuminuria.

[symbol] multicenter, randomized, double-blind, placebo-controlled, parallel-designed

1-2. Grounds for Clinical Trial Design

(1) Grounds for Subject Selection

The study drug is to be administered twice a day to type 2 diabetic nephropathy patients with albuminuria, the test group of this clinical trial.

Diabetes and hypertension are significant risk factors for chronic kidney disease, and diabetic nephropathy, a chronic complication of diabetes, is particularly evaluated as a major cause of chronic kidney disease. The KDIGO (Kidney Disease Improving Global Outcomes) guideline recommends a target blood pressure of less than 140/90 mmHg for people whose urine albumin excretion is 30 mg/g Cr or less as a target blood pressure to reduce cardiovascular disease-related deaths and suppress the progression of nephropathy. In addition, a lower target blood pressure of less than 130/80 mmHg is recommended for people with a urine albumin excretion of 30 mg/g Cr or more or for people at high risk of cardiovascular disease. According to the Korean diabetes treatment guideline, it is recommended to control the blood sugar of diabetic patients to less than 6.5% of HbA1c. In diabetes, blood pressure control is important to lower the risk of cardiovascular disease and to suppress the development and progression of diabetic nephropathy. Therefore, the present inventors intend to conduct this clinical trial on patients whose blood pressure is controlled under 140/90 mmHg by taking ACE inhibitors or ARB agents.

(2) Grounds for Control Group Selection

As a control group for this clinical trial, placebo is to be administered twice a day to diabetic nephropathy patients with albuminuria.

It was judged that it was not appropriate to completely exclude treatment for the disease by administering only a placebo in consideration of safety and clinical practice environment. Therefore, subjects who have been taking ACE inhibitors and ARBs for at least 2 months prior to screening were selected, and it was attempted to ensure safety by allowing the subjects to take a placebo while receiving maintenance treatment for the disease.

(3) Grounds for Dosage Setting

By referring to the previously approved dose and usage of Tecfidera® capsule, which has already been approved as one of the drugs to delay the course of the disease in multiple sclerosis, and is used worldwide, the present inventors intend to explore the optimal dose for the treatment of diabetic nephropathy.

The efficacy of DMF was evaluated in the UUO-induced renal fibrosis mouse model and the streptozotocin (STZ, 150 mg/kg)-induced diabetic nephropathy mouse model using the drug of the present invention. As a result, it was confirmed that a dose of 25 mg/kg was effective for diabetic nephropathy.

When the dose confirmed in the animal model is converted into HED (Human Equivalent Dose), it is calculated as follows.

HED=Animal dose in mg/kg×{Animal weight in kg/Human weight in kg}×60 kg=25 mg/kg×0.08×60 kg=120 mg

Therefore, in this clinical trial, it is expected to be effective when administered more than 120 mg.

DMF is hydrolyzed by esterase and converted to monomethyl fumarate (MMF), which is an active metabolite. When administered orally, DMF is not detected in plasma. It is known that the half-life of DMF in human serum is confirmed to be about 12 minutes, and the elimination half-life of MMF is as short as about 1 hour. It is known that MMF does not accumulate in the body even after repeated administration and is not detected 24 hours after administration of the drug.

Therefore, in this clinical trial, referring to the results of the non-clinical study, the short half-life, and the dose and usage of the previously approved Tecfidera® capsule, it was decided to administer 120 mg twice a day, once a day in order to expect continued efficacy for the disease.

(4) Grounds for Efficacy Evaluation Variable Setting

Microalbuminuria can be evaluated by measuring thereof in urine collected for a certain period (24-hour or overnight collected urine) and spot urine. Dipstick test, a general urine test, is not recommended for the evaluation of albuminuria in diabetic patients. The reason is that microalbuminuria is not detected by the dipstick test, and the results of the quantification of albuminuria vary depending on the patient's hydration status and various factors, so albuminuria measurement is not accurate. In general, although quantitative analysis of albuminuria through 24-hour collected urine is the most common method, there is a problem in the inconvenience of collecting urine and the accuracy of urine collection. Overnight timed urine collection can be used as an alternative method to compensate for this disadvantage, but the sensitivity of albuminuria quantification may low because the urine collection period is short. Due to various problems, the American Diabetes Association and Kidney Association recommend using the Albumin/Creatinine ratio (spot urine ACR) in urine using spot urine.

The American Diabetes Association recommends measuring the changes in microalbuminuria and glomerular filtration rate (GFR) annually for the evaluation of renal function in patients with type 2 diabetes. However, although most diabetic patients are expected to accompany a large amount of albuminuria, in many patients, research results have been recently published that there is no microalbuminuria and only renal function is reduced. 55% of diabetic patients with renal dysfunction showed no microalbuminuria. However, renal dysfunction patients without microalbuminuria showed a higher frequency in the general population than in diabetic patients, regardless of the patient's gender, race, and duration of diabetes. Several hypotheses have been proposed as the cause of diabetic patients with reduced renal function without microalbuminuria. First, there is a possibility that albuminuria may have disappeared due to the effects of renin-angiotensin (RAS) blockers in use in most patients, blood pressure control by the use of blood pressure drugs, and the effects of hyperlipidemia drugs. Another possibility is that complications of diabetes mainly occur in the tubular interstitial tissue and may be accompanied by non-diabetic kidney disease, which may be accompanied by diabetes. It may also appear as a result of a complex interaction between diseases of renal blood vessels invading the afferent arterioles and the rapid progression of the aging process of the kidneys. However, there is currently no study on the pathophysiological mechanism of these patients, so more active research is needed on the pathophysiology and treatment of diabetic chronic kidney disease not accompanied by albuminuria. In addition, in patients diagnosed with diabetes, renal function may decrease even in the absence of microalbuminuria, and periodic GFR measurement must be performed together.

For this reason, the present inventors intend to use it as a major variable for this indication.

There are few studies on the relationship between chronic kidney disease and β-cell function. It may differ depending on the presence or absence of type 2 diabetes due to differences in β cell function or mass. Diabetes is a common cause of chronic kidney disease, and the function and mass decrease due to the clinical onset of type 1 diabetes and type 2 diabetes. This is accompanied by a decrease in glycemic control, and some studies have suggested that β cells function to alter the disease course of type 2 diabetes.

According to a prior literature (DeFronzo, Ralph A., Roy Eldor, and Muhammad Abdul-Ghani. “Pathophysiologic approach to therapy in patients with newly diagnosed type 2 diabetes.” Diabetes care 36. Supplement 2 (2013): S127-S138), it was reported that cell dysfunction is a common pathological feature of type 1 and type 2 diabetes, and type 2 diabetes does not develop without β cell dysfunction. In addition, the above literature suggested that the restoration of β cell function should be the primary treatment target for type 2 diabetes.

In the group with type 2 diabetes, the prevalence of chronic kidney disease is higher than in the group without type 2 diabetes. As the cause of these results, compensatory action is thought to occur in the group without type 2 diabetes, and restoration of β cell function in diabetic patients is considered to be helpful in preventing or delaying the progression of chronic kidney disease.

In this clinical trial, referring to a study that confirmed the relationship between β-cell function and chronic renal disease in patients with type 2 diabetes, HOMA-β was used as an index to evaluate the relationship with diabetic nephropathy, one of the renal diseases.

C-peptide, an active peptide hormone, has the potential to cause major physiological effects. C-peptide is produced in the same amount as insulin, and it attenuates glomerular hyperfiltration and reduces albumin excretion. C-peptide is the best indicator (sign) of endogenous insulin secretion in diabetic patients, and the amount of C-peptide in the blood represents the amount of insulin, which is produced in the pancreas. The blood glucose level of the body is not affected by C-peptide, and it is used to determine whether type 1 diabetes or type 2 diabetes is in the early diagnosis of diabetes.

In a study by Rebsomen et al. (Rebsomen, L., et al. “C-Peptide effects on renal physiology and diabetes.” Experimental diabetes research 2008), theories related to C-peptide on renal function were reviewed and reported that the therapeutic role of C-peptide as a protective factor for diabetic kidney should not be ignored. In a study by Chowta et al. [14], the correlation between C-peptide levels, renal clearance, urinary albumin excretion, and duration of diabetes was investigated. According to the results, it was found that there was a correlation between serum C-peptide, microalbuminuria and creatinine clearance, and that the risk of albuminuria was increased in patients with low C-peptide levels. In addition, in some studies (Maimoona Mushtaq Masoom, et al. “C-Peptide as a Marker for Diabetic Nephropathy” OMICS International, Intern Med, an open access journal (2017): Volume 7 issue 3), C-peptide and renal involvement in diabetes were investigated, and it was found that there was a significant correlation between C-peptide, microalbumin-creatinine ratio and microalbumin, and there was a significant difference between Cystatin C and C-peptide levels.

However, these studies have a limitation in that the number of patients is very small, and emphasized the need for additional research.

Therefore, in this clinical trial, the effectiveness of diabetic nephropathy using C-peptide as an indicator is to be confirmed by referring to the above studies.

2. Clinical Trial Method

2-1. Overall Clinical Trial Method

[symbol] multicenter, randomized, double-blind, placebo-controlled, parallel-designed

In this clinical trial, an investigational drug is taken for 12 weeks, and its efficacy and safety are evaluated.

The investigational drug is delivered at the baseline (Visit 2, Day 0) and start taking from the morning of Day 1. All subjects visit at Visit 3 (phone visit), Visit 4 (6 weeks post-administration), Visit 5 (EOT+1 day: 12 weeks post-administration), and Visit 6 (follow-up). wherein, subjects who agree to the pharmacokinetic test visit the testing laboratory on Day 1 and the day before Visit 5 (EOT) and perform the pharmacokinetic test according to the procedure.

Subjects visit for final evaluation the next day after taking the last investigational drug (Visit 5).

After 6 weeks of safety follow-up, the clinical trial is terminated.

2-2. Administration Dose, Administration Method and Administration Period of Investigational Drug

Forty type 2 diabetic nephropathy patients with albuminuria are randomly assigned to a test group and a control group, 20 each. The investigational drug is administered for 12 weeks with a double-blind condition.

Each administration group should be taken with food and administered twice a day (morning and evening).

Test Group

[symbol] Investigational drug: Example 11 (dimethyl fumarate 120 mg/tablet), twice a day, 2 tablets in total

Control Group

[symbol] Control drug: placebo, twice a day, 2 tablets in total

At Visit 2 (baseline, Day 0), the investigational drug according to the test group and control group is randomly prescribed. The investigational drug distributed at Visit 2 is taken from the morning of the next day of prescription (Day 1) and returned after taking by the morning of Visit 4. The investigational drug distributed at Visit 4 is taken from the evening of the prescription date to the day before the last visit (EOT).

3. Evaluation Criteria, Evaluation Method

3-1. Evaluation Criteria and Evaluation Method of Efficacy Evaluation Variables

(1) Changes in ACR (Albumin to Creatinine Ratio)

The amount of changes in the urine albumin-creatinine ratio at 6 weeks and 12 weeks after the administration compared to the baseline for each administration group is evaluated.

ACR=Urine albumin concentration×1,000/Urine creatinine concentration

The definitions of microalbuminuria and overt albuminuria are shown in table 6 below.

TABLE 6

| | Albumin excretion | | |
|---|---|---|---|
| | mg/24 h urine | μg/min. (min) | μg/mg (creatinine) |
| Normal | <30 | <20 | <30 |
| Microalbuminuria | 30~299 | 20~199 | 30~299 |
| Overt albuminuria | ≥300 | ≥200 | ≥300 |

(2) Changes in GFR (Glomerular Filtration Rate)

The amount of changes in GFR at 6 weeks and 12 weeks after the administration compared to the baseline for each administration group is evaluated. GFR is evaluated as eGFR (estimated GFR) value using the IDMS-MDRD formula derived from the concentration of serum creatinine.

The corresponding derivation formula is as follows.
IDMS-MDRD formula:
Male

eGFR (ml/min/1.73 m²)=175×(serum creatinine)^{-1.154}×(age)^{-0.203}
Female

eGFR (ml/min/1.73 m²)=175×(serum creatinine)^{-1.154}×(age)^{-0.203}×0.742

(3) Changes in C-Peptide

The amount of changes in C-peptide at 6 weeks and 12 weeks after the administration compared to the baseline for each administration group is evaluated.

(4) Changes in Transforming Growth Factor Beta1 (TGF-β1)

The amount of changes in serum transforming growth factor beta1 (TGF-β1) at the end of the administration (12 weeks) compared to the baseline for each administration group is evaluated.

3-2. Evaluation Criteria and Evaluation Method of Safety Evaluation Variables

(1) Adverse Reaction

In order to evaluate safety, adverse reactions for the entire period of the clinical trial are confirmed.

(2) Laboratory Test

Laboratory tests are performed at every visit except for the telephone visit (Visit 3), and blood collection is performed on an empty stomach (at least 8 hours). The tests are performed by collecting the first urine at the institution on the day of the visit or at least 2 hours after eating. The relevant items of laboratory tests are as follows. However, at the follow-up visit (Visit 6), only hematological and hemochemical tests are performed at the local laboratory.
Hematological test: RBC, WBC, Hemoglobin, Hematocrit, Platelets count, WBC Differential count (Eosinophil, Basophil, Neutrophil, Lymphocyte, Monocyte)
Hemochemical test: Na, K, Ca, Cl, Mg, P, BUN, Creatinine, Uric acid, Total bilirubin, Albumin, Total protein, Creatine kinase, ALT, AST, γ-GTP, Alkaline phosphatase, Glucose, Insulin, Total cholesterol, HDL Cholesterol, LDL Cholesterol, Triglyceride, LDH, HbA1c, C-peptide
Urine test: Protein, Glucose, Occult blood, Ketones, pH, Specific gravity, Bilirubin, Urobilinogen, Nitrite, Albumin, Creatinine

Screening and laboratory tests to assess subject suitability at baseline are performed using the local laboratory. Laboratory tests for the evaluation of subject suitability at the baseline may use the results obtained from screening.

Central laboratory results are used for the laboratory tests (Urine Albumin, Urine creatinine, Serum Creatinine, Fasting plasma insulin, Fasting plasma glucose, C-peptide) to evaluate the efficacy of subjects (ACR, GFR, HOMA-β, C-peptide) for the treatment period, and all laboratory test items are collected using local laboratories for timely safety evaluation.

Abnormalities and findings in laboratory tests that are judged to be clinically significant after administration of the investigational drug are recorded as adverse reactions. Laboratory tests confirmed as adverse reactions are followed up using the laboratory test results of each institution (central laboratory or local laboratory) that has been discovered and confirmed.

With the collected laboratory test specimens (blood and urine), additional analysis can be planned in addition to the analysis planned in this protocol. If applicable, it should be implemented only when a separate consent form has been obtained for each subject.

(3) Electrocardiography

After 12 weeks of screening and administration, electrocardiography is performed and the results are checked. At the time of screening, if there are test results performed within the past 4 weeks from the date of the visit, they can be used. However, if the heart rate is irregular or serious changes are observed, a retest can be performed at the examiner's discretion. Abnormal values and findings in electrocardiography that are judged to be clinically significant after administration of the investigational drug are recorded as adverse reactions.

(4) Vital Signs

Vital signs are measured at all visits except for the telephone visit (Visit 3). Blood pressure, pulse, and body temperature (tympanic membrane) are measured, and blood pressure and pulse rate are measured after resting in a sitting position for at least 5 minutes. Abnormal values and findings in vital signs judged to be clinically significant that occurred after administration of the investigational drug are recorded as adverse reactions.

(5) Physical Examination

Physical examination is performed at all visits except for the telephone visit (Visit 3) and the follow-up visit (Visit 6). Abnormal findings on physical examination that are judged to be clinically significant after administration of the investigational drug are recorded as adverse reactions.

3-3. Evaluation Criteria and Evaluation Methods of Pharmacokinetic Tests

The tablet of Example 11 is rapidly hydrolyzed and converted to MMF, the active metabolite, after oral administration. Therefore, since DMF cannot be quantified in plasma after oral administration, analysis of plasma MMF is performed. CL, V_{d}, Cₘₐₓ, Tₘₐₓ, AUCₗₐₛₜ, AUC_{inf}, AUCₑₓₜᵣₐ and t_{1/2}, the pharmacokinetic parameters of plasma MMF before and after administration of the tablet of Example 11 are evaluated.

Pharmacokinetic tests are performed on a total of 6 subjects (test group: 5, control group: 1). For the pharmacokinetic evaluation of plasma MMF at the start date of administration (1D) and 12 weeks after administration, the values of the parameters to be evaluated are analyzed by time points for the start date of administration and 12 weeks after administration, respectively. The time points of blood sampling are as follows.
Before administration (Window time—15 min.)
After administration: 0.5, 1, 1.5, 1.75, 2, 2.5, 3, 3.5, 4, 6, 8 hr (Window time±15 min.)

The 6 subjects undergoing the pharmacokinetic study should visit the first day of administration (1D) and 12 weeks after administration of the investigational drug (EOT). Pharmacokinetic tests are performed according to the procedure before and after taking the investigational drug in the morning of the day.

4. Efficacy Evaluation

4-1. Primary Variables for Efficacy Evaluation 1) Changes in ACR at the end of administration for 12 weeks compared to baseline

4-2. Secondary Variables for Efficacy Evaluation

1) Changes in ACR at 6 weeks compared to baseline

2) Changes in GFR at 6 weeks and at the end of administration for 12 weeks compared to baseline

3) Changes in HOMA-β at 6 weeks and at the end of administration for 12 weeks compared to baseline

4) Changes in C-peptide at 6 weeks and at the end of administration for 12 weeks compared to baseline

4-3. Exploratory Variables for Efficacy Evaluation

1) Changes in transforming growth factor beta1 (TGF-β1) at the end of administration for 12 weeks compared to baseline

5. Safety Evaluation

5-1. Variables for Safety Evaluation

1) Adverse event

2) Laboratory test

3) Electrocardiography

4) Vital signs

5) Physical examination

6. Pharmacokinetic Evaluation

6-1. Variables for Pharmacokinetic Evaluation

Pharmacokinetic parameters of plasma MMF at the start date of administration of the tablet of Example 11 (1D) and 12 weeks after administration: CL, V_{d}, Cₘₐₓ, Tₘₐₓ, AUCₗₐₛₜ, AUC_{inf}, AUCₑₓₜᵣₐ, t_{1/2}

7. Statistical Analysis Method

7-1. Variables for Efficacy Evaluation

(1) Primary Variables for Efficacy Evaluation

Descriptive statistics (mean value, standard deviation, median value, minimum value and maximum value) on the amount of changes in ACR at 12 weeks after administration compared to the baseline for each administration group are presented, and a 95% two-sided confidence interval based on t-distribution for differences between administration groups is presented.

(2) Secondary Variables for Efficacy Evaluation

(2-A) Changes in ACR at 6 weeks compared to baseline: Descriptive statistics (mean value, standard deviation, median value, minimum value and maximum value) on the amount of changes in ACR at 6 weeks after administration compared to the baseline for each administration group are presented, and whether there is a difference between the administration groups is analyzed using two sample t-test (If normality assumption is not satisfied, Wilcoxon's rank sum test is used).

(2-B) Changes in GFR at 6 weeks and at the end of administration for 12 weeks compared to baseline: Descriptive statistics (mean value, standard deviation, median value, minimum value and maximum value) on the amount of changes in GFR at 6 weeks and at the end of administration for 12 weeks compared to the baseline for each administration group are presented, and whether there is a difference between the administration groups is analyzed using two sample t-test (If normality assumption is not satisfied, Wilcoxon's rank sum test is used).

(2-C) Changes in HOMA-β at 6 weeks and at the end of administration for 12 weeks compared to baseline: Descriptive statistics (mean value, standard deviation, median value, minimum value and maximum value) on the amount of changes in HOMA-β at 6 weeks and at the end of administration for 12 weeks compared to the baseline for each administration group are presented, and whether there is a difference between the administration groups is analyzed using two sample t-test (If normality assumption is not satisfied, Wilcoxon's rank sum test is used).

(2-D) Changes in C-peptide at 6 weeks and at the end of administration for 12 weeks compared to baseline: Descriptive statistics (mean value, standard deviation, median value, minimum value and maximum value) on the amount of changes in C-peptide at 6 weeks and at the end of administration for 12 weeks compared to the baseline for each administration group are presented, and whether there is a difference between the administration groups is analyzed using two sample t-test (If normality assumption is not satisfied, Wilcoxon's rank sum test is used).

7-2. Variables for Safety Evaluation

Safety analysis is performed on the subject of safety evaluation.

(1) Adverse Reaction

Summarization and analysis of adverse reactions are performed on the adverse reactions occurred after administration of the investigational drug. The frequency and percentage of adverse events (AE), adverse drug reactions (ADR), and serious adverse events (SAE) occurring after administration of the investigational drug are presented, and the difference between the administration groups is analyzed using Pearson's chi-square test. If the number of cells with an expected frequency of less than 5 exceeds 20%, Fisher's exact test is used for analysis. Adverse events are coded according to System Organ Class and Preferred Term using MedDRA (Medical Dictionary for Regulatory Activities: latest version), and the number of subjects, incidence rates and occurrences of the coded adverse events are presented for each administration group.

(2) Laboratory Test, Electrocardiography, Vital Signs, Physical Examination

For continuous variables, descriptive statistics (mean value, standard deviation, median value, minimum value and maximum value) are presented for each visit and analyzed using paired t-test (If normality assumption is not satisfied, Wilcoxon's signed rank test is used) to confirm the changes within the administration group. In order to confirm the difference between the administration groups, comparative analysis is conducted using two-sample t-test (If normality assumption is not satisfied, Wilcoxon's rank sum test is used). In the case of categorical variables, frequency and percentage are presented, and McNemar's test is used to confirm the changes within the administration group, and Pearson's chi-square test is used (If the number of cells with an expected frequency of less than 5 exceeds 20%, Fisher's exact test is used) to confirm the difference between the administration groups.

8. Grounds for Setting the Number of Subjects

This test is a clinical trial to confirm the possibility of entering a therapeutic confirmation trial, unlike the typical test for statistical hypothesis testing, for the purpose of evaluating the dose response after identifying safety and efficacy characteristics after administering the tablet of Example 11 to type 2 diabetic nephropathy patients with albuminuria. Therefore, in order to confirm the possibility of entering a therapeutic confirmation trial, the number of subjects is determined without considering statistical significance. The target number of test subjects for this study is 16 per group, and a total of 40 subjects will be enrolled at 20 per group considering 20% of dropouts, of which 6 subjects will be subjected to pharmacokinetic analysis (However, when considering additional registration of replacement subjects due to dropout of pharmacokinetic test subjects, more than 40 subjects can be enrolled).

9. Clinical Trial Results

The clinical trial results derived through the aforementioned clinical trial design and method are as follows.
Test group: (investigational drug) Example 11 (dimethyl fumarate 120 mg/tablet), twice a day, 2 tablets in total
Control group: (placebo), twice a day, 2 tablets in total

9-1. Efficacy Evaluation

(1) Primary Efficacy Evaluation

a. Changes in ACR at the end of administration for 12 weeks compared to baseline—FAS

TABLE 7

| | |
|---|---|
| Test group | Control group |
| (N = 20) | (N = 19) |

Change at Week 12 + Day 1 from Baseline

| | | |
|---|---|---|
| Mean ± SD | −60.30 ± 197.22 | −59.96 ± 159.38 |
| P-value^{[A]} | 0.0441 | 0.1336 |
| P-value^{[B]} | 0.5838 | — |

Treatment Difference

| | |
|---|---|
| Mean ± SD | −0.34 ± 179.81 |

[A] In-group comparison: Paired t-test[P] or Wilcoxon's signed rank test[S]

[B] Comparison between groups: Two sample t-test[T] or Wilcoxon's rank sum test[W]

As shown in table 7, the ACR value of the test group was decreased by an average of 60.30 at the end of the 12-week administration compared to the baseline, and the p-value of the change amount within the group was 0.0441, showing a statistically significant difference. On the other hand, the ACR value of the control group was decreased by an average of 59.96, and the p-value of the change amount within the group was 0.1336, and there was no statistically significant difference. Compared to the control group, the ACR value of the test group was decreased by an average of 0.34, and the p-value of the change amount between the groups was 0.5838, and there was no statistically significant difference.

(2) Secondary Efficacy Evaluation

a. Changes in GFR at 6 weeks and at the end of administration for 12 weeks compared to baseline—FAS

TABLE 8

| | |
|---|---|
| Test group | Control group |
| (N = 20) | (N = 19) |

Change at Week 6 from Baseline

| | | |
|---|---|---|
| Mean ± SD | 2.35 ± 7.70 | −2.37 ± 5.51 |
| P-value^{[A]} | 0.1882 | 0.0773 |
| P-value^{[B]} | 0.0349 | — |

Change at Week 12 + Day 1 from Baseline

| | | |
|---|---|---|
| Mean ± SD | 3.80 ± 8.72 | −4.63 ± 11.14 |
| P-value^{[A]} | 0.0663 | 0.0867 |
| P-value^{[B]} | 0.0121 | — |

[A] In-group comparison: Paired t-test[P] or Wilcoxon's signed rank test[S]

[B] Comparison between groups: Two sample t-test[T] or Wilcoxon's rank sum test[W]

As shown in table 8, the amount of changes in GFR at 6 weeks and at the end of administration for 12 weeks compared to the baseline was increased by an average of 2.35 and 3.80 in the test group, respectively, and decreased by an average of 2.37 and 4.63 in the control group, respectively. The differences in GFR changes between the administration groups at 6 weeks and 12 weeks were 4.72 and 8.43, respectively, which were statistically significant differences (p-value=0.0349, 0.0121).

b. Changes in C-peptide at 6 weeks and at the end of administration for 12 weeks compared to baseline—FAS

TABLE 9

| | |
|---|---|
| Test group | Control group |
| (N = 20) | (N = 19) |

Change at Week 6 from Baseline

| | | |
|---|---|---|
| Mean ± SD | −0.09 ± 0.59 | 0.02 ± 0.57 |
| P-value^{[A]} | 0.5060 | 0.9047 |
| P-value^{[B]} | 0.5731 | — |

Change at Week 12 + Day 1 from Baseline

| | | |
|---|---|---|
| Mean ± SD | 0.08 ± 0.61 | −0.12 ± 0.78 |
| P-value^{[A]} | 0.5911 | 0.5253 |
| P-value^{[B]} | 0.3998 | — |

[A] In-group comparison: Paired t-test[P] or Wilcoxon's signed rank test[S]

[B] Comparison between groups: Two sample t-test[T] or Wilcoxon's rank sum test[W]

As shown in table 9, the amount of changes in C-peptide at 6 weeks and at the end of administration for 12 weeks compared to the baseline was decreased by an average of 0.09 and increased by 0.08 in the test group, respectively, and increased by an average of 0.02 and decreased by 0.12 in the control group, respectively. The differences in C-peptide changes between the administration groups at 6 weeks and 12 weeks were 0.11 and 0.20, respectively, which were not statistically significant differences (p-value=0.5731, 0.3998).

(3) Exploratory Variables for Efficacy Evaluation

a. Changes in transforming growth factor beta1 (TGF-β1) at the end of administration for 12 weeks compared to baseline

TABLE 10

| Screening | Random | Test group | Screening | Random | Control group |
|---|---|---|---|---|---|
| No. | No. | (CU01-1001) | No. | No. | (Placebo) |
| S01-013 | R01-207 | 2588.8 | S01-004 | R01-201 | −2762.7 |
| S01-017 | R01-211 | −15907.4 | S01-006 | R01-203 | 857.6 |
| S02-001 | R02-201 | −3147.9 | S01-010 | R01-206 | −7917.8 |
| S03-001 | R03-101 | −8459.3 | S01-015 | R01-209 | −18.7 |
| S03-004 | R03-102 | −2616.4 | S01-016 | R01-210 | −3467.8 |
| S03-009 | R03-104 | −2677.4 | S02-003 | R02-203 | 756.2 |
| S03-012 | R03-105 | −11963.3 | S03-006 | R03-201 | 16329.4 |
| S03-013 | R03-203 | 957.5 | S03-008 | R03-103 | 8533.5 |
| S03-015 | R03-204 | 1234.6 | S04-003 | R04-201 | 11353.7 |
| S04-006 | R04-203 | −5503.7 | S04-004 | R04-202 | 3592 |
| S04-007 | R04-204 | −7206.9 | S04-008 | R04-205 | 4281.8 |
| S04-012 | R04-208 | 788.1 | S04-009 | R04-206 | 1810.2 |
| S04-015 | R04-210 | −1321.6 | S04-010 | R04-207 | 13233.1 |
| S04-016 | R04-211 | −9620.1 | S04-017 | R04-212 | 4419.3 |
| S04-019 | R04-214 | 140.5 | S04-018 | R04-213 | −627.7 |
| S06-007 | R06-202 | 2036 | S04-020 | R04-215 | 2115.4 |

| | | | |
|---|---|---|---|
| Average | −3792.40 | Average | 3280.46 |

As shown in table 10, the average value of changes in transforming growth factor beta1 (TGF-β1) at the end of administration for 12 weeks in the test group was −3792.40 compared to the baseline, and it was decreased in most of the test subjects. On the other hand, the average value of changes in transforming growth factor beta1 (TGF-β1) in the control group was 3280.46, and it was increased in most of the test subjects.

9-2. Safety Evaluation

Adverse Event

Status of adverse event (AE) occurred after administration of investigational drug—Safety Set

TABLE 11

| | Test group | Control group |
|---|---|---|
| | (N = 22) | (N = 19) |
| Adverse event (AE) | 9 (40.91%) | 4 (21.05%) |
| P-value^{[B]} | 0.1730 | — |
| Adverse drug reaction (ADR) | 8 (36.36%) | 1 (5.26%) |
| P-value^{[B]} | 0.0238 | — |
| Serious adverse event (SAE) | 1 (4.55%) | 0 (0.00%) |
| P-value^{[B]} | 1.0000 | — |
| Serious adverse drug reaction (SADR) | 0 (0.00%) | 0 (0.00%) |
| P-value^{[B]} | — | — |

As shown in table 11, after administration of the investigational drug, adverse event (AE) occurred in 9 out of 22 subjects (40.91%) in the test group and 4 out of 19 subjects (21.05%) in the control group, and the p-value indicating the difference between groups was 0.1730, which was not statistically significant difference. Adverse drug reaction (ADR) occurred in 8 out of 22 subjects (36.36%) in the test group and 1 out of 19 subjects (5.26%) in the control group, and the p-value indicating the difference between groups was 0.0238, which was statistically significant. Serious adverse event (SAE) occurred in 1 out of 22 subjects (4.55%) in the test group, not occurred in the control group (0/19, 0.00%), and the p-value indicating the difference between groups was 1.0000, which was not statistically significant difference. Serious adverse drug reaction (SADR) did not occur in either the test group or the control group.

Classification of adverse event (AE) occurred after administration of investigational drug—Safety Set

TABLE 12

| | |
|---|---|
| Test group | Control group |
| (N = 22) | (N = 19) |

| | | | | |
|---|---|---|---|---|
| Gastrointestinal disorders) | 4 | (18.18%) | 3 | (15.79%) |
| Nausea | 3 | (13.64%) | 0 | (0.00%) |
| Diarrhea | 0 | (0.00%) | 2 | (10.53%) |
| Lower abdominal pain | 0 | (0.00%) | 1 | (5.26%) |
| Epigastric pain | 1 | (4.54%) | 0 | (0.00%) |
| Vascular disorders | 4 | (18.18%) | 0 | (0.00%) |
| Blush | 3 | (13.64%) | 0 | (0.00%) |
| Heat sensation | 1 | (4.54) | 0 | (0.00%) |

Classification of adverse drug reaction (ADR) occurred after administration of investigational drug—Safety Set

TABLE 13

| | |
|---|---|
| Test group | Control group |
| (N = 22) | (N = 19) |

| | | | |
|---|---|---|---|
| Gastrointestinal disorders | 3 | (13.64%) | 1 (5.26%) |
| Nausea | 2 | (9.09%) | 0 (0.00%) |
| Diarrhea | 0 | (0.00%) | 1 (5.26%) |
| Epigastric pain | 1 | (4.55%) | 0 (0.00%) |
| Vascular disorders | 4 | (18.18%) | 0 (0.00%) |
| Blush | 3 | (13.64%) | 0 (0.00%) |
| Heat sensation | 1 | (4.55%) | 0 (0.00%) |

As shown in tables 12 and 13, the most frequent adverse events were blush (including facial blush) and nausea (18.18% and 13.64%, respectively). On the other hand, no serious adverse drug reaction that could not be excluded from the administration of Example 11 was observed, confirming the safety and tolerability of Example 11.

9-3. Pharmacokinetic Evaluation

The pharmacokinetic evaluation results (LLOQ: 10 ng/mL, ULOQ: 2500 ng/mL) are shown in table 14 below.

TABLE 14

| | Parameter | | | | | |
|---|---|---|---|---|---|---|
| | Cₘₐₓ | Tₘₐₓ | AUCₗₐₛₜ | AUC_{inf} | AUCₑₓₜᵣₐ | t_{1/2} |
| Number | (ng/mL) | (hr) | (ng*hr/mL) | (ng*hr/mL) | (%) | (hr) |
| N | 6 | 6 | 6 | 6 | 6 | 6 |
| Mean | 1675.6 | 0.8 | 1908.0 | 1936.1 | 1.4 | 0.8 |
| SD | 669.7 | 0.6 | 370.6 | 377.2 | 0.7 | 0.1 |
| % CV¹⁾ | 40.0 | 81.6 | 19.4 | 19.5 | 46.9 | 15.2 |
| Median | 1695.3 | 0.5 | 1784.7 | 1811.7 | 1.5 | 0.8 |
| Min | 927.3 | 0.5 | 1547.6 | 1567.2 | 0.5 | 0.6 |
| Max | 2811.5 | 2.0 | 2378.7 | 2421.6 | 2.3 | 0.9 |

¹⁾Coefficient of variation (%) = (SD/Mean) *100

What is claimed is:

1. A method for preventing or treating diabetic nephropathy, comprising a step of administering a pharmaceutical composition containing 60 to 480 mg of dimethyl fumarate or monomethyl fumarate or a pharmaceutically acceptable salt thereof as an active ingredient, and the administration of the pharmaceutical composition to a subject provides one or more of the following pharmacokinetic parameters:
(a) Mean plasma monomethyl fumarate Cₘₐₓ of 1675.6 ng/mL (±10%);
(b) Mean plasma monomethyl fumarate Tₘₐₓ of 0.8 hr (±10%);
(c) Mean plasma monomethyl fumarate AUCₗₐₛₜ of 1908.0 hr·ng/mL (±10%);
(d) Mean plasma monomethyl fumarate AUC_{inf} of 1936.1 hr·ng/mL (±10%);
(e) Mean plasma monomethyl fumarate AUCₑₓₜᵣₐ of 1.4% (±10%);
(f) Mean plasma monomethyl fumarate t_{1/2} of 0.8 hr (±10%);
wherein, each of the parameters of (a) to (f) is a value when 120 mg of the active ingredient is included, and the active ingredient shows a dose-proportional linear elimination kinetics.

2. The method according to claim 1, wherein the pharmaceutical composition contains 110 to 250 mg of dimethyl fumarate or monomethyl fumarate or a pharmaceutically acceptable salt thereof as an active ingredient, and it is administered twice a day.

3. The method according to claim 1, wherein the pharmaceutical composition contains 115 to 125 mg of dimethyl fumarate or monomethyl fumarate or a pharmaceutically acceptable salt thereof as an active ingredient, and it is administered twice a day.

4. The method according to claim 1, wherein the method prevents, alleviates or treats kidney fibrosis symptoms.

5. The method according to claim 1, wherein the pharmaceutical composition is provided in the form of an enteric-coated tablet containing a core comprising dimethyl fumarate or monomethyl fumarate or a pharmaceutically acceptable salt thereof as an active ingredient; an enteric coating layer; and a seal coating layer comprising a cellulose-based polymer between the core and the enteric coating layer, in which the enteric coating layer is included in an amount of 6 to 9 weight part based on 100 weight part of the core, the seal coating layer is included in an amount of 1 to 3 weight part based on 100 weight part of the core, and the particle size distribution of the dimethyl fumarate or monomethyl fumarate or a pharmaceutically acceptable salt thereof satisfies one or more of the following conditions:
(a) The mean particle size of the lower 90% of the particles (D90) is 100 μm or less;
(b) The mean particle size of the lower 50% of the particles (D50) is 50 μm or less; and
(c) The mean particle size of the lower 10% of the particles (D10) is 20 μm or less.

6. The method according to claim 5, wherein the active ingredient is included in an amount of 20 to 60 weight % based on the core.

7. The method according to claim 5, wherein the core contains at least one pharmaceutically acceptable additive selected from the group consisting of excipients, disintegrants and lubricants.

8. The method according to claim 7, wherein the excipient is included in an amount of 30 to 45 weight %, the disintegrant is included in an amount of 10 to 20 weight %, and the lubricant is included in an amount of 0.1 to 2 weight % based on the core.

9. The method according to claim 5, wherein the core further includes an alkalizing agent.

10. The method according to claim 9, wherein the weight ratio of the alkalizing agent and the active ingredient is 0.5:12 to 2:12.

11. The method according to claim 9, wherein the alkalizing agent is included in an amount of 2 to 5 weight % based on the core.

12. The method according to claim 9, wherein the alkalizing agent is Meglumine or a pharmaceutically acceptable salt thereof.

13. The method according to claim 5, wherein the enteric coating layer comprises at least one enteric coating polymer selected from the group consisting of an enteric acrylic acid-based copolymer selected from the group consisting of styrene-acrylic acid copolymer, ethyl methacrylate methacrylate copolymer, methyl acrylate methacrylate octyl acrylate copolymer, and ethyl methacrylate acrylate copolymer; an enteric cellulose-based polymer selected from the group consisting of hydroxypropylmethylcellulose acetate succinate, hydroxypropylmethylcellulose phthalate, hydroxymethylethylcellulose phthalate, cellulose acetate phthalate, cellulose acetate maleate, cellulose acetate succinate, cellulose acetate maleate, cellulose benzoate phthalate, cellulose propionate phthalate, methylcellulose phthalate, carboxymethylethylcellulose, ethylhydroxyethylcellulose phthalate, carboxymethylethylcellulose and ethylhydroxyethylcellulose phthalate; an enteric maleic acid-based copolymer selected from the group consisting of vinyl acetate maleic acid anhydride copolymer, styrene maleic acid anhydride copolymer, styrene maleic acid monoesterol copolymer, vinylmethyl ether maleic acid anhydride copolymer, ethylene maleic acid anhydride copolymer, vinyl butyl ether maleic acid anhydride copolymer, acrylonitrile methyl acrylate maleic acid anhydride copolymer and butyl acrylate styrene maleic acid anhydride copolymer; and an enteric polyvinyl-based polymer selected from the group consisting of polyvinyl alcohol phthalate, polyvinyl acetacetal phthalate, polyvinyl butyrate phthalate and polyvinyl acetacetal phthalate.

14. The method according to claim 5, wherein the thickness of the coating layer of the enteric-coated tablet is 20 μm to 90 μm.
